(19) United States
(12) Patent Application Publication
(10) Pub. No.: US 2015/0110889 A1
(43) Pub. Date: Apr. 23, 2015

(54) COSMETIC COMPOSITION COMPRISING A GEMINI SURFACTANT AND HIGH LEVELS OF SOLID FATTY SURFACTANTS

(71) Applicant: L'OREAL, Paris (FR)

(72) Inventors: Raluca Lorant, Thiais (FR); Florence Lahousse, Thiais (FR)

(21) Appl. No.: 14/352,778

(22) PCT Filed: Oct. 16, 2012
(86) PCT No.: PCT/EP2012/070513

§ 371 Date: Apr. 18, 2014

Related U.S. Application Data
(60) Provisional application No. 61/551,519, filed on Oct. 26, 2011.

(30) Foreign Application Priority Data
Oct. 21, 2011 (FR) 1159555

(51) Int. Cl. A61K 8/46; A61Q 19/00; A61Q 19/08

(52) U.S. Cl. CPC A61K 8/466; A61Q 19/08; A61Q 19/00; A61K 2800/10
USPC 424/539; 514/552

(57) ABSTRACT

A subject of the present invention is a cosmetic composition of oil-in-water emulsion type comprising: (1) at least one gemini surfactant of formula (I):

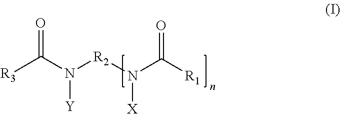

in which R₁ and R₃ denote, independently of one another, an alkyl radical having from 1 to 25 carbon atoms; R₂ denotes a spacing group consisting of a linear or branched alkylene chain having from 1 to 12 carbon atoms; X and Y denote, independently of one another, a —(C₂H₄O)ₐ—(C₃H₆O)_{b}Z group; n ranges from 1 to 10; and -n ranges from 1 to 10; and (2) at least 20% by weight of the total weight of the composition of a fatty phase comprising at least one oil and at least one fatty substance chosen from solid fatty substances and pasty fatty substances; the level of fatty substances chosen from solid fatty substances and pasty fatty substances representing between ⅓ and ⅔ by weight of the fatty phase. The composition in accordance with the invention has a very significant consistency, ranging from balm to butter, or even to solid emulsions (sticks), original and pleasant sensory properties, such as a melting away effect, easy application, a non-greasy and non-shiny finish, and also good stability.

The invention relates to a cosmetic composition of oil-in-water emulsion type intended for keratin materials, in particular the skin and the lips, the hair and the nails. The invention also relates to a cosmetic method for treating keratin materials using said composition.

Textures rich in solid fatty substances and having high viscosities (such as butters) are particularly advantageous for skin care. Indeed, they are particularly recommended for caring for mature, dehydrated skin exhibiting lipid deficiencies. They provide nutritive and soothing effects and persistent moisturization. On the other hand, by virtue of their high viscosity (consistency), they are difficult to spread, and penetrate slowly while leaving the skin greasy and shiny. Moreover, when the level of solid fatty substances (waxy or pasty) is high (greater than 5%), the emulsions are not very stable and pose crystallization or phase separation problems.

There remains therefore the need for products which are in the form of thick balms or butters which exhibit a melting away effect on application, which would allow easy and pleasant spreading, and which do not leave a greasy shiny film at the surface of the skin.

The applicant has discovered that this need can be satisfied by combining, in a composition of emulsion type, a gemini surfactant and at least 20% by weight of particular fatty phase comprising solid fatty substances and/or pasty fatty substances.

More specifically, a subject of the present invention is a cosmetic composition of oil-in-water emulsion type comprising:

(1) at least one gemini surfactant of formula (I):

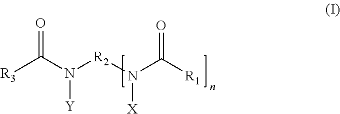

in which:
R₁ and R₃ denote, independently of one another, an alkyl radical having from 1 to 25 carbon atoms;
R₂ denotes a spacing group consisting of a linear or branched alkylene chain having from 1 to 12 carbon atoms;
X and Y denote, independently of one another, a —(C₂H₄O)ₐ—(C₃H₆O)_{b}Z group, where:
   Z denotes a hydrogen atom or a —CH₂—COOM, —SO₃M, —P(O)(OM)₂, —C₂H₄—SO₃M, —C₃H₆—SO₃M or —CH₂(CHOH)₄CH₂OH radical, where M represents H or an alkali metal or alkaline earth metal or ammonium or alkanolammonium ion,
   a ranges from 0 to 15,
   b ranges from 0 to 10, and
   the sum of a+b ranges from 1 to 25; and
n ranges from 1 to 10; and

(2) at least 20% by weight of the total weight of the composition of a fatty phase comprising at least one oil and at least one fatty substance chosen from solid fatty substances and pasty fatty substances;

the level of fatty substances chosen from solid fatty substances and pasty fatty substances representing between ⅓ and ⅔ by weight of the fatty phase.

As the composition of the invention is intended for topical application to the skin or superficial body growths, it comprises a physiologically acceptable medium, that is to say a medium compatible with all keratin materials, such as the skin, nails, mucous membranes and keratin fibres (such as the hair or eyelashes).

The composition in accordance with the invention has a very significant consistency, ranging from balm to butter, or even to solid emulsions (sticks), original and pleasant sensory properties, such as a melting away effect, easy application, a non-greasy and non-shiny finish, and also good stability.

Another subject of the present invention is a cosmetic method for making up and/or caring for keratin materials comprising a step of applying a composition as defined above to said materials.

In that which follows and unless otherwise indicated, the limits of a range of values are included in this range.

According to one particular embodiment, the composition in accordance with the invention has a viscosity, measured at 25° C. using a Rheomat RM 180 viscometer equipped with a No. 4 rotor, after rotation of the rotor in the composition for 1 min at a speed of 200 revolutions/min, of between 45 and 220 poises.

Gemini Surfactant

The gemini surfactant of formula (I) is preferably such that each of the R₁—CO— and R₃—CO— groups comprises from 8 to 20 carbon atoms, and preferably denotes a coconut fatty acid residue (comprising mainly lauric acid and myristic acid).

In addition, this surfactant is preferably such that, for each of the X and Y radicals, the sum of a and b has an average value ranging from 10 to 20 and is preferably equal to 15. A preferred group for Z is the —SO₃M group, where M is preferably an alkali metal ion, such as a sodium ion.

The spacing group R₂ advantageously consists of a linear C₁-C₃ alkylene chain, and preferably an ethylene (CH₂CH₂) chain.

Finally, n is advantageously equal to 1.

A surfactant of this type is in particular the one identified by the INCI name: Sodium dicocoylethylenediamine PEG-15 sulfate, having the following structure:

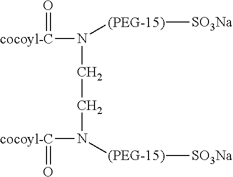

it being understood that PEG represents the CH₂CH₂O group, and cocoyl represents the coconut fatty acid residue.

This surfactant has a molecular structure very similar to that of ceramide-3.

Preferably, the gemini surfactant according to the invention is used as a mixture with other surfactants, and in particular as a mixture with (a) an ester of a C₆-C₂₂ fatty acid (preferably C₁₄-C₂₀ such as a stearate) and of glyceryl, (b) a diester of a C₆-C₂₂ fatty acid (preferably C₁₄-C₂₀ such as a stearate) and of citric acid and of glycerol (in particular a diester of a C₆-C₂₂ fatty acid and of glyceryl monocitrate), and (c) a C₁₀-C₃₀ fatty alcohol (preferably behenyl alcohol).

Advantageously, the composition according to the invention comprises a mixture of sodium dicocoylethylenediamine PEG-15 sulfate, of glyceryl stearate, of glyceryl stearate monocitrate, of behenyl alcohol.

More preferentially, the gemini surfactant according to the invention represents from 10 to 20% by weight, and advantageously 15% by weight; the ester of a C₆-C₂₂ fatty acid and of glyceryl represents from 30 to 40% by weight, advantageously 35% by weight; the diester of a C₆-C₂₂ fatty acid and of citric acid and of glycerol represents from 10 to 20% by weight, advantageously 15% by weight; and the C₁₀-C₃₀ fatty alcohol represents from 30 to 40% by weight, advantageously 35% by weight, relative to the total weight of the mixture of surfactants containing the gemini surfactant.

Advantageously, the composition according to the invention comprises a mixture of from 10 to 20% by weight of sodium dicocoylethylenediamine PEG-15 sulfate, from 30 to 40% (in particular 35%) by weight of glyceryl stearate, from 10 to 20% (in particular 15%) by weight of glyceryl stearate monocitrate, and from 30 to 40% (in particular 35%) by weight of behenyl alcohol, relative to the total weight of the mixture of surfactants containing the gemini surfactant.

As a variant, the gemini surfactant according to the invention can be used as a mixture with an anionic surfactant, such as an ester of lauric acid, sodium lauroyl lactate. In this case, the gemini surfactant preferably represents from 30 to 50% by weight, and the anionic surfactant represents from 50 to 70% by weight, relative to the total weight of the mixture.

The gemini surfactant may be used, for example, as a mixture with other surfactants in the form of the products sold by the company Sasol under the name Ceralution®, and in particular the following products:
Ceralution® H: Behenyl Alcohol, Glyceryl Stearate, Glyceryl Stearate Citrate and Sodium Dicocoylethylenediamine PEG-15 Sulfate,
Ceralution® F: Sodium Lauroyl Lactylate and Sodium Dicocoylethylenediamine PEG-15 Sulfate,
Ceralution® C: Aqua, Capric/Caprylic triglyceride, Glycerin, Ceteareth-25, Sodium Dicocoylethylenediamine PEG-15 Sulfate, Sodium Lauroyl Lactylate, Behenyl Alcohol, Glyceryl Stearate, Glyceryl Stearate Citrate, Gum Arabic, Xanthan Gum, Phenoxyethanol, Methylparaben, Ethylparaben, Butylparaben, Isobutylparaben (INCI names).

This gemini surfactant represents from 3 to 50% of the weight of these mixtures.

The gemini surfactant of formula (I) may be present in a composition according to the invention in a content ranging from 0.01% to 5% by weight, preferably ranging from 0.1% to 3% by weight and better still ranging from 0.2% to 1.5% by weight relative to the total weight of the composition.

Fatty Phase

The proportion of the fatty phase may range, for example, from 20% to 50% by weight, preferably from 20% to 45% by weight and better still from 20% to 40% by weight relative to the total weight of the composition.

This amount indicated does not include the content of lipophilic surfactants.

For the purpose of the invention, the fatty phase includes any fatty substance which is liquid at ambient temperature and atmospheric pressure, generally oils, or which is solid at ambient temperature and atmospheric pressure, like waxes, or any pasty compound, which are present in said composition.

The fatty phase of the composition in accordance with the invention comprises at least one fatty substance chosen from solid fatty substances and pasty fatty substances. According to one particular embodiment of the invention, the solid fatty substance(s) is (are) chosen from waxes.

For the purpose of the present invention, the term “pasty fatty substance” is intended to mean a lipophilic fatty compound that undergoes a reversible solid/liquid change in state, that exhibits an anisotropic crystal organization in the solid state, and that comprises, at a temperature of 23° C., a liquid fraction and a solid fraction.

In other words, the starting melting point of the pasty fatty substance can be less than 23° C. The liquid fraction of the pasty fatty substance measured at 23° C. can represent from 9% to 97% by weight of the pasty fatty substance. This liquid fraction at 23° C. preferably represents between 15% and 85% and more preferably between 40% and 85% by weight.

For the purpose of the invention, the melting point corresponds to the temperature of the most endothermic peak observed on thermal analysis (DSC) as described in Standard ISO 11357-3; 1999. The melting point of a pasty fatty substance may be measured using a differential scanning calorimeter (DSC), for example the calorimeter sold under the name MDSC 2920 by the company TA Instruments.

The measuring protocol is as follows:

A sample of 5 mg of pasty fatty substance placed in a crucible is subjected to a first temperature rise ranging from −20° C. to 100° C., at a heating rate of 10° C./minute, is then cooled from 100° C. to −20° C. at a cooling rate of 10° C./minute and is finally subjected to a second temperature rise ranging from −20° C. to 100° C. at a heating rate of 5° C./minute. During the second temperature rise, the variation in the difference in power absorbed by the empty crucible and by the crucible containing the sample of pasty fatty substance is measured as a function of the temperature. The melting point of the pasty fatty substance is the value of the temperature corresponding to the tip of the peak of the curve representing the variation in the difference in power absorbed as a function of the temperature.

The liquid fraction by weight of the pasty fatty substance at 23° C. is equal to the ratio of the enthalpy of fusion consumed at 23° C. to the enthalpy of fusion of the pasty fatty substance.

The enthalpy of fusion of the pasty fatty substance is the enthalpy consumed by the latter in order to pass from the solid state to the liquid state. The pasty fatty substance is said to be in the solid state when all of its mass is in crystalline solid form. The pasty fatty substance is said to be in the liquid state when all of its mass is in liquid form.

The enthalpy of fusion of the pasty fatty substance is equal to the area under the curve of the thermogram obtained using a differential scanning calorimeter (DSC), such as the calorimeter sold under the name MDSC 2920 by the company TA Instruments, with a temperature rise of 5° C. or 10° C. per minute, according to standard ISO 11357-3:1999.

The enthalpy of fusion of the pasty fatty substance is the amount of energy required to make the pasty fatty substance change from the solid state to the liquid state. It is expressed in J/g. The enthalpy of fusion consumed at 23° C. is the amount of energy absorbed by the sample to change from the solid state to the state that it has at 23° C., composed of a liquid fraction and a solid fraction.

The liquid fraction of the pasty fatty substance measured at 32° C. preferably represents from 30% to 100% by weight of the pasty fatty substance, preferably from 50% to 100%, more preferably from 60% to 100% by weight of the pasty fatty substance. When the liquid fraction of the pasty fatty substance measured at 32° C. is equal to 100%, the temperature of the end of the melting range of the pasty fatty substance is less than or equal to 32° C.

The liquid fraction of the pasty fatty substance measured at 32° C. is equal to the ratio of the enthalpy of fusion consumed at 32° C. to the enthalpy of fusion of the pasty fatty substance. The enthalpy of fusion consumed at 32° C. is calculated in the same way as the enthalpy of fusion consumed at 23° C.

The pasty fatty substance is preferably chosen from synthetic fatty substances and fatty substances of vegetable origin. A pasty fatty substance may be obtained by synthesis from starting materials of vegetable origin.

The pasty fatty substance is advantageously chosen from:
lanolin and derivatives thereof,
polyol ethers chosen from pentaerythrityl ethers of a polyalkylene glycol, fatty alkyl ethers of a sugar, and mixtures thereof, the pentaerythrityl ether of polyethylene glycol comprising 5 oxyethylene units (5 OE) (CTFA name: PEG-5 Pentaerythrityl Ether), the pentaerythrityl ether of polypropylene glycol comprising 5 oxypropylene units (5 OP) (CTFA name: PPG-5 Pentaerythrityl Ether), and mixtures thereof, and more especially the mixture PEG-5 Pentaerythrityl Ether, PPG-5 Pentaerythrityl Ether and soybean oil, sold under the name Lanolide by the company Vevy, which is a mixture in which the constituents are in a 46/46/8 weight ratio: 46% PEG-5 Pentaerythrityl Ether, 46% PPG-5 Pentaerythrityl Ether and 8% soybean oil,
polymeric or non-polymeric silicone compounds,
polymeric or non-polymeric fluoro compounds,
vinyl polymers, especially:
   olefin homopolymers and copolymers,
   hydrogenated diene homopolymers and copolymers,
   linear or branched oligomers which are homopolymers or copolymers of alkyl (meth)acrylates preferably containing a C₈-C₃₀ alkyl group,
   oligomers which are homopolymers and copolymers of vinyl esters containing C₈-C₃₀ alkyl groups,
   oligomers which are homopolymers and copolymers of vinyl ethers containing C₈-C₃₀ alkyl groups,
liposoluble polyethers resulting from the polyetherification between one or more C₂-C₁₀₀ and preferably C₂-C₅₀ diols,
esters,
and/or mixtures thereof.

The pasty fatty substance is preferably a polymer, in particular a hydrocarbon-based polymer.

Among the liposoluble polyethers that are particularly preferred are copolymers of ethylene oxide and/or of propylene oxide with C₆-C₃₀ long-chain alkylene oxides, more preferably such that the weight ratio of the ethylene oxide and/or of the propylene oxide to the alkylene oxides in the copolymer is from 5:95 to 70:30. In this family, mention will in particular be made of copolymers such that the long-chain alkylene oxides are arranged in blocks having an average molecular weight from 1000 to 10000, for example a polyoxyethylene/polydodecyl glycol block copolymer such as the ethers of dodecanediol (22 mol) and of polyethylene glycol (45 OE) sold under the brand name Elfacos ST9 by Akzo Nobel.

Among the esters, the following are in particular preferred:
esters of a glycerol oligomer, especially diglycerol esters, in particular condensates of adipic acid and of glycerol, for which some of the hydroxyl groups of the glycerols have reacted with a mixture of fatty acids such as stearic acid, capric acid, isostearic acid and 12-hydroxystearic acid, especially such as the product sold under the brand name Softisan 649 by the company Sasol,
the arachidyl propionate sold under the brand name Waxenol 801 by Alzo,
phytosterol esters,
fatty acid triglycerides and derivatives thereof,
pentaerythritol esters,
esters of a diol dimer and of a diacid dimer, where appropriate esterified on their free alcohol or acid functional group(s) with acid or alcohol radicals, especially dimer dilinoleate esters; such esters may be chosen especially from the esters having the following INCI nomenclature: bis-behenyl/isostearyl/phytosteryl dimer dilinoleyl dimer dilinoleate (Plandool G), phytosteryl isostearyl dimer dilinoleate (Lusplan PI-DA, Lusplan PHY/IS-DA), phytosteryl/isostearyl/cetyl/stearyl/behenyl dimer dilinoleate (Plandool H or Plandool S), and mixtures thereof,
mango butter, such as the product sold under the reference Lipex 203 by the company AarhusKarlshamn,
hydrogenated soybean oil, hydrogenated coconut oil, hydrogenated rapeseed oil, mixtures of hydrogenated vegetable oils such as the mixture of hydrogenated soybean, coconut, palm and rapeseed vegetable oil, for example the mixture sold under the reference Akogel® by the company AarhusKarlshamn (INCI name: Hydrogenated Vegetable Oil),
shea butter, in particular the product for which the INCI name is Butyrospermum Parkii Butter, such as the product sold under the reference Sheasoft® by the company AarhusKarlshamn,
cocoa butter, in particular the product which is sold under the name CT Cocoa Butter Deodorized by the company Dutch Cocoa BV or the product which is sold under the name Beurre De Cacao NCB HD703 758 by the company Barry Callebaut,
shorea butter, in particular the product which is sold under the name Dub Shorea T by the company Stearinerie Dubois,
and mixtures thereof.

According to one preferred embodiment, the pasty fatty substance is chosen from a mixture of hydrogenated soybean, coconut, palm and rapeseed vegetable oils, shea butter, cocoa butter, shorea butter, and mixtures thereof, and more particularly those referenced above.

The waxes under consideration in the context of the present invention are generally lipophilic compounds that are solid and deformable or non-deformable at ambient temperature (25° C.), with a solid/liquid reversible change of state, having a melting point of greater than or equal to 30° C., which may range up to 200° C. and in particular up to 120° C.

By bringing one or more wax(es), in accordance with the invention, to the liquid state (melting), it is possible to render it (them) miscible with one or more oils and to form a macroscopically homogeneous wax(es)+oil(s) mixture, but if the temperature of said mixture is returned to ambient temperature, recrystallization of the wax(es) in the oil(s) of the mixture is obtained.

For the purpose of the invention, the melting point corresponds to the temperature of the most endothermic peak observed on thermal analysis (DSC) as described in Standard ISO 11357-3; 1999. The melting point of the wax may be measured using a differential scanning calorimeter (DSC), for example the calorimeter sold under the name MDSC 2920 by the company TA Instruments.

The measuring protocol is as follows:

A sample of 5 mg of wax placed in a crucible is subjected to a first temperature rise ranging from −20° C. to 100° C., at a heating rate of 10° C./minute, it is then cooled from 100° C. to −20° C. at a cooling rate of 10° C./minute and it is finally subjected to a second temperature rise ranging from −20° C. to 100° C. at a heating rate of 5° C./minute. During the second temperature rise, the variation in the difference in power absorbed by the empty crucible and by the crucible containing the sample of wax is measured as a function of the temperature. The melting point of the compound is the temperature value corresponding to the top of the peak of the curve representing the variation in the difference in power absorbed as a function of the temperature.

The waxes that may be used in a composition according to the invention are chosen from waxes, that are solid at ambient temperature, of animal, vegetable, mineral or synthetic origin, and mixtures thereof. They may be hydrocarbon-based, fluoro and/or silicone waxes.

By way of examples, mention may in particular be made of hydrocarbon-based waxes, such as natural beeswax (or bleached beeswax), synthetic beeswax, carnauba wax, rice bran wax, such as the product sold under the reference NC 1720 by the company Cera Rica Noda, candelilla wax, such as the product sold under the reference SP 75 G by the company Strahl & Pitsch, microcrystalline waxes, for instance the microcrystalline waxes of which the melting point is above 85° C., such as the products HI-MIC® 1070, 1080, 1090 and 3080 sold by the company Nippon Seiro, ceresins or ozokerites, for instance isoparaffins of which the melting point is below 40° C., such as the product EMW-0003 sold by the company Nippon Seiro, α-olefin oligomers, such as the Performa V® 825, 103 and 260 polymers sold by the company New Phase Technologies; ethylene/propylene copolymers, such as Performalene® EP 700, polyethylene waxes (preferably having a molecular weight of between 400 and 600), Fischer-Tropsch waxes, the sunflower seed wax sold by the company Koster Keunen under the reference sunflower wax.

Mention may also be made of silicone waxes, for instance alkyl or alkoxy dimethicones containing from 16 to 45 carbon atoms, and fluoro waxes.

According to one particular embodiment, the wax used in a composition in accordance with the invention has a melting point above 35° C., better still above 40° C., or even above 45° C. or above 55° C.

According to one preferred embodiment, the wax(es) is (are) chosen from polymethylene waxes; the silicone wax sold under the name Dow Corning 2501 Cosmetic Wax by the company Dow Corning (INCI name: bis-peg-18 methyl ether dimethyl silane); beeswax; vegetable waxes, such as carnauba wax; the mixture of polyglycerolated (3 mol) vegetable (mimosa/jojoba/sunflower) waxes sold under the name Hydracire S by the company Gattefosse, the hydrogenated castor oil sold under the name Antisettle CVP by the company Cray Valley.

According to one particular embodiment of the invention, the fatty phase comprises at least one wax, such as carnauba wax or polymethylene wax, at least one pasty fatty substance, such as cocoa butter, and at least one oil.

According to one particular embodiment of the invention, the level of fatty substances chosen from the solid fatty substances and the pasty fatty substances is at least 10% by weight, preferably between 10% and 30% by weight, and even more preferentially between 10% and 20% by weight relative to the total weight of the composition.

The fatty phase of the composition in accordance with the invention also comprises at least one oil. The oil(s) present in the composition may be volatile or non-volatile.

The term “oil” means any fatty substance that is in liquid form at ambient temperature (25° C.) and at atmospheric pressure.

The volatile or non-volatile oils may be hydrocarbon-based oils, in particular of animal or vegetable origin, synthetic oils, silicone oils or fluoro oils, or mixtures thereof.

For the purposes of the present invention, the term “silicone oil” means an oil comprising at least one silicon atom, and in particular at least one Si—O group.

The term “hydrocarbon-based oil” means an oil mainly containing hydrogen and carbon atoms, and optionally oxygen, nitrogen, sulfur and/or phosphorus atoms.

Non-Volatile Oils

For the purposes of the present invention, the term “non-volatile oil” means an oil having a vapour pressure of less than 0.13 Pa (0.01 mmHg).

The non-volatile oils may be chosen in particular from non-volatile hydrocarbon-based oils, which may be fluorinated, and/or non-volatile silicone oils.

As non-volatile hydrocarbon-based oils that are suitable for use in the invention, mention may be made in particular of:
hydrocarbon-based oils of animal origin,
hydrocarbon-based oils of vegetable origin, such as phytostearyl esters, such as phytostearyl oleate, phytostearyl isostearate and lauroyl/octyl-dodecyl/phytostearyl glutamate, for example sold under the name Eldew PS203 by Ajinomoto, triglycerides consisting of fatty acid esters of glycerol, the fatty acids of which may have chain lengths ranging from C₄ to C₂₄, these chains possibly being linear or branched, and saturated or unsaturated; these oils are in particular heptanoic or octanoic triglycerides, wheatgerm oil, sunflower oil, grapeseed oil, sesame oil, corn oil, apricot oil, castor oil, shea oil, avocado oil, olive oil, soybean oil, sweet almond oil, palm oil, rapeseed oil, cotton seed oil, hazelnut oil, macadamia oil, jojoba oil, alfalfa oil, poppyseed oil, pumpkin oil, marrow oil, blackcurrant oil, evening primrose oil, millet oil, barley oil, quinoa oil, rye oil, safflower oil, candlenut oil, passion flower oil or musk rose oil; shea butter; or alternatively caprylic/capric acid triglycerides, for instance those sold by the company Stearineries Dubois or those sold under the names Miglyol 810®, 812® and 818® by the company Dynamit Nobel, the refined vegetable perhydrosqualene sold under the name Fitoderm by the company Cognis;
hydrocarbon-based oils of mineral or synthetic origin, for instance:
   synthetic ethers containing from 10 to 40 carbon atoms;
   linear or branched hydrocarbons of mineral or synthetic origin, such as petroleum jelly, polydecenes, hydrogenated polyisobutene such as Parleam, and squalane, and mixtures thereof, and in particular hydrogenated polyisobutene;
   synthetic esters, for instance oils of formula R₁COOR₂ in which R₁ represents a linear or branched fatty acid residue containing from 1 to 40 carbon atoms and R₂ represents a hydrocarbon-based chain that is in particular branched, containing from 1 to 40 carbon atoms provided that R₁+R₂ is ≧10.

The esters may be chosen in particular from fatty acid esters, for instance:
cetostearyl octanoate, isopropyl alcohol esters, such as isopropyl myristate, isopropyl palmitate, ethyl palmitate, 2-ethylhexyl palmitate, isopropyl stearate, isopropyl isostearate, isostearyl isostearate, octyl stearate, hydroxylated esters, for instance isostearyl lactate, octyl hydroxystearate, diisopropyl adipate, heptanoates, and in particular isostearyl heptanoate, alcohol or polyalcohol octanoates, decanoates or ricinoleates, for instance propylene glycol dioctanoate, cetyl octanoate, tridecyl octanoate, 2-ethylhexyl 4-diheptanoate, 2-ethylhexyl palmitate, alkyl benzoate, polyethylene glycol diheptanoate, propylene glycol 2-diethylhexanoate, and mixtures thereof, C₁₂-C₁₅ alcohol benzoates, hexyl laurate, neopentanoic acid esters, for instance isodecyl neopentanoate, isotridecyl neopentanoate, isostearyl neopentanoate, octyldodecyl neopentanoate, isononanoic acid esters, for instance isononyl isononanoate, isotridecyl isononanoate, octyl isononanoate, hydroxylated esters, for instance isostearyl lactate and diisostearyl malate;
polyol esters and pentaerythritol esters, for instance dipentaerythrityl tetrahyd roxystearate/tetraisostearate;
esters of diol dimers and of diacid dimers, such as Lusplan DD-DA5® and Lusplan DD-DA7® sold by the company Nippon Fine Chemical and described in patent application FR 0302809;
fatty alcohols that are liquid at ambient temperature, with a branched and/or unsaturated carbon-based chain containing from 12 to 26 carbon atoms, for instance 2-octyldodecanol, isostearyl alcohol, oleyl alcohol, 2-hexyldecanol, 2-butyloctanol and 2-undecylpentadecanol;
higher fatty acids such as oleic acid, linoleic acid and linolenic acid, and mixtures thereof; and
dialkyl carbonates, the two alkyl chains possibly being identical or different, such as the dicaprylyl carbonate sold under the name Cetiol CC® by Cognis;
non-volatile silicone oils, for instance non-volatile polydimethylsiloxanes (PDMSs), polydimethylsiloxanes comprising alkyl or alkoxy groups that are pendent and/or at the end of a silicone chain, these groups each containing from 2 to 24 carbon atoms, phenyl silicones, for instance phenyl trimethicones, phenyl dimethicones, phenyltrimethylsiloxydiphenylsiloxanes, diphenyl dimethicones, diphenylmethyld iphenyltrisiloxanes and 2-phenylethyl trimethylsiloxysilicates, and dimethicones or phenyl trimethicones with a viscosity of less than or equal to 100 cSt, and mixtures thereof;
and mixtures thereof.

Volatile Oils

For the purposes of the present invention, the term “volatile oil” means an oil (or non-aqueous medium) that is capable of evaporating on contact with the skin in less than one hour, at ambient temperature and at atmospheric pressure. The volatile oil is a volatile cosmetic oil, which is liquid at ambient temperature, especially having a non-zero vapour pressure, at ambient temperature and atmospheric pressure, in particular having a vapour pressure ranging from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

The volatile hydrocarbon-based oils may be chosen from hydrocarbon-based oils containing from 8 to 16 carbon atoms, and in particular branched C₈-C₁₆ alkanes (also known as isoparaffins), for instance isododecane (also known as 2,2,4,4,6-pentamethylheptane), isodecane, isohexadecane and, for example, the oils sold under the trade names Isopar® or Permethyl®.

Volatile oils that may also be used include volatile silicones, for instance volatile linear or cyclic silicone oils, in particular those with a viscosity 8 centistokes (8×10⁻⁶ m²/s), and especially containing from 2 to 10 silicon atoms and in particular from 2 to 7 silicon atoms, these silicones optionally comprising alkyl or alkoxy groups containing from 1 to 10 carbon atoms. As volatile silicone oils that may be used in the invention, mention may be made in particular of dimethicones with viscosities of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Volatile fluoro oils such as nonafluoromethoxybutane or perfluoromethylcyclopentane, and mixtures thereof, may also be used.

It is also possible to use a mixture of the oils mentioned above.

The other fatty substances that may be present in the fatty phase are, for example, fatty acids containing from 8 to 30 carbon atoms, for instance stearic acid, lauric acid or palmitic acid; fatty alcohols containing from 8 to 30 carbon atoms, for instance stearyl alcohol or cetyl alcohol and mixtures thereof (cetearyl alcohol).

The fatty phase may also contain other compounds dissolved in the oils, such as gelling agents and/or structuring agents.

These compounds may in particular be chosen from gums, such as silicone gums (dimethiconol); silicone resins, such as trifluoromethyl(C₁-C₄ alkyl)dimethicone and trifluoropropyl dimethicone, and silicone elastomers, for instance the products sold under the KSG names by the company Shin-Etsu, under the name Trefil by the company Dow Corning or under the Gansil names by the company Grant Industries; and mixtures thereof.

These fatty substances may be chosen in a varied manner by a person skilled in the art so as to prepare a composition having the desired properties, for example of consistency or texture.

According to one particular embodiment, the composition in accordance with the invention comprises the combination either of at least one polar wax, such as carnauba wax, with at least one apolar oil, such as isohexadecane, or of at least one apolar wax, such as polymethylene wax, with a polar oil, such as isononyl isononanoate.

These combinations make it possible to structure the fatty phase in order to obtain textures which at the same time are solid and firm and melt away.

For the purposes of the present invention, the term “polar oil” means an oil of which the solubility parameter δₐ at 25° C. is other than 0 (J/cm³)^{1/2}.

In particular, the term “polar oil” means an oil of which the chemical structure is formed essentially from, or even consists of, carbon and hydrogen atoms, and comprising at least one highly electronegative heteroatom such as an oxygen, nitrogen, silicon or phosphorus atom.

The definition and calculation of the solubility parameters in the Hansen three-dimensional solubility space are described in the article by C. M. Hansen: “The three dimensionnal solubility parameters” J. Paint Technol. 39, 105 (1967).

According to this Hansen space:
δ_{D} characterizes the London dispersion forces derived from the formation of dipoles induced during molecular impacts;
δₚ characterizes the Debye interaction forces between permanent dipoles and also the Keesom interaction forces between induced dipoles and permanent dipoles;
δₕ characterizes the specific interaction forces (such as hydrogen bonding, acid/base, donor/acceptor, etc.); and
δₐ is determined by the equation: δₐ=(δₚ²+δₕ²)^{1/2}.

The parameters δₚ, δₕ, δ_{D} and δₐ are expressed in (J/cm³)^{1/2}.

For the purposes of the present invention, the term “apolar oil” means an oil of which the solubility parameter δₐ at 25° C. as defined above is equal to 0 (J/cm³)^{1/2}.

By way of examples, mention may be made of the combination of beeswax with parleam (or hydrogenated polyisobutylene), the combination of microcrystalline wax with pentaerythrityl tetraisostearate, or the combination of candelilla wax with vegetable perhydrosqualene, such as the product which is sold under the name Fitoderm by the company Cognis.

Aqueous Phase

The aqueous phase of the composition according to the invention comprises at least water.

According to the galenical form of the composition, the amount of aqueous phase can range from 30% to 80% by weight, preferably from 35% to 70% by weight and better still from 40% to 60% by weight relative to the total weight of the composition. This amount depends on the galenical form of the composition desired. The amount of water can represent all or a portion of the aqueous phase and it is generally at least 30% by weight relative to the total weight of the composition.

The aqueous phase can comprise at least one hydrophilic solvent, such as, for example, substantially linear or branched lower monoalcohols having from 1 to 8 carbon atoms, such as ethanol, propanol, butanol, isopropanol or isobutanol; polyols, such as propylene glycol, isoprene glycol, butylene glycol, glycerol, sorbitol, polyethylene glycols and derivatives thereof; and mixtures thereof.

In a known manner, all the compositions of the invention can contain one or more of the adjuvants that are customary in the cosmetic and dermatological fields, hydrophilic or lipophilic gelling agents and/or thickeners; moisturisers; emollients; hydrophilic or lipophilic active agents; free radical scavengers; sequestering agents; antioxidants; preservatives; basifying or acidifying agents; fragrances; film-forming agents; colorants (pigments such as iron oxides and titanium dioxide), pearlescent agents, soluble dyes, fillers; and mixtures thereof.

The amounts of these various adjuvants are those conventionally used in the fields under consideration. In particular, the amounts of active agents vary according to the desired objective and are those conventionally used in the fields under consideration, and for example from 0.1% to 20%, and preferably from 0.5% to 10% of the total weight of the composition.

Hydrophilic gelling agents that may be mentioned include, for example, carboxyvinyl polymers such as carbopols (carbomers) and the Pemulens (acrylate/C₁₀-C₃₀ alkyl acrylate copolymer); polyacrylamides, for instance the crosslinked copolymers sold under the names Sepigel 305 (CTFA name: polyacrylamide/C13-14 isoparaffin/Laureth 7) or Simulgel 600 (CTFA name: acrylamide/sodium acryloyldimethyltaurate copolymer/isohexadecane/polysorbate 80) by the company Seppic; cellulose derivatives such as hydroxyethylcellulose; polysaccharides and in particular gums such as xanthan gum; and mixtures thereof.

Lipophilic gelling agents that may be mentioned include modified clays such as hectorite and derivatives thereof, for instance the products sold under the name Bentone.

Fillers

According to one particular embodiment, the composition in accordance with the invention comprises at least one matting filler.

As matting fillers which can be used in the composition of the invention, mention may, for example, be made of silicas such as the polymer with the INCI name Methylsilanol/Silicate Crosspolymer sold under the name NLK 506 by the company Takemoto Oil & Fat; silica such as the silica microspheres sold under the name SB 700 by the company Miyoshi Kasei; kaolin; talc; boron nitride; organic spherical powders, fibres; and mixtures thereof. Examples of organic spherical powders that may be mentioned include polyamide powders and especially Nylon® powders such as Nylon-1 or Polyamide 12, sold under the name Orgasol by the company Atochem; polyethylene powders; Teflon®; microspheres based on acrylic copolymers, such as those made of ethylene glycol dimethacrylate/lauryl methacrylate copolymer, sold by the company Dow Corning under the name Polytrap; expanded powders such as hollow microspheres and especially the microspheres sold under the name Expancel by the company Kemanord Plast or under the name Micropearl F 80 ED by the company Matsumoto; silicone resin microbeads such as those sold under the name Tospearl by the company Toshiba Silicone; polymethyl methacrylate microspheres, sold under the name Microsphere M-100 by the company Matsumoto or under the name Covabead LH85 by the company Wackherr; ethylene acrylate copolymer powders, such as those sold under the name Flobeads by the company Sumitomo Seika Chemicals; powders of natural organic materials such as starch powders, especially of maize starch, wheat starch or rice starch, which may or may not be crosslinked, such as the starch powders crosslinked with octenyl succinate anhydride, sold under the name Dry-Flo by the company National Starch. Examples of fibres that may be mentioned include polyamide fibres, such as in particular Nylon 6 (or Polyamide 6) (INCI name: Nylon 6) fibres, Nylon 6,6 (or Polyamide 66) (INCI name: Nylon 66) fibres, or such as poly-p-phenyleneterephthamide fibres; and mixtures thereof.

These fillers may be present in amounts ranging from 0% to 20% by weight, preferably from 0.5% to 10% by weight and even more preferentially from 0.5% to 5% by weight relative to the total weight of the composition.

Active Agents

By way of example of an active agent, mention may be made, in a nonlimiting manner, of ascorbic acid and derivatives thereof such as 5,6-di-O-dimethylsilyl ascorbate (sold by the company Exsymol under the reference PRO-AA), the potassium salt of dl-alpha-tocopheryl-2l-ascorbyl phosphate (sold by the company Senju Pharmaceutical under the reference Sepivital EPC), magnesium ascorbyl phosphate, sodium ascorbyl phosphate (sold by the company Roche under the reference Stay-C 50); phloroglucinol; enzymes; and mixtures thereof. Among the hydrophilic active agents sensitive to oxidation, ascorbic acid is used according to one preferred embodiment of the invention. The ascorbic acid may be of any nature. Thus, it may be of natural origin in powder form or in the form of orange juice, preferably orange juice concentrate. It may also be of synthetic origin, preferably in powder form.

As other active agents that can be used in the composition of the invention, mention may be made, for example, of moisturising agents, such as protein hydrolysates and polyols, for instance glycerol, glycols, for instance polyethylene glycols; natural extracts; anti-inflammatories; oligomeric proanthocyanidins; vitamins such as vitamin A (retinol), vitamin E (tocopherol), vitamin B5 (panthenol), vitamin B3 (niacinamide), derivatives of these vitamins (in particular esters) and mixtures thereof; urea; caffeine; depigmenting agents such as kojic acid, hydroquinone and caffeic acid; salicylic acid and derivatives thereof; alpha-hydroxy acids, such as lactic acid and glycolic acid and derivatives thereof; retinoids, such as carotenoids and vitamin A derivatives; hydrocortisone; melatonin; extracts of algae, of fungi, of plants, of yeasts, of bacteria; steroids; antibacterial active agents, such as 2,4,4′-trichloro-2′-hydroxy diphenyl ether (or triclosan), 3,4,4′-trichlorocarbanilide (or triclocarban) and the acids indicated above, and in particular salicylic acid and derivatives thereof; matting agents, for instance fibres; tensioning agents; UV-screening agents; and mixtures thereof.

Needless to say, a person skilled in the art will take care to select the optional adjuvant(s) added to the composition according to the invention such that the advantageous properties intrinsically associated with the composition in accordance with the invention are not, or are not substantially, adversely affected by the envisaged addition.

The composition according to the invention is in the form of an oil-in-water emulsion, of liquid or semi-liquid consistency of the milk type for example, obtained by dispersion of a fatty phase in an aqueous phase, or of suspensions or emulsions of soft, semi-solid or solid consistency of the cream or gel type. These compositions are prepared according to the usual methods.

In addition, the composition in accordance with the invention may be more or less fluid and may have the appearance of a gel, a white or coloured cream, an ointment, a milk, a lotion, a serum, a paste or a mousse.

The composition preferably has a skin-friendly pH which generally ranges from 3 to 8 and preferably from 4.5 to 7.

The examples that follow will allow the invention to be understood more clearly, without, however, being limiting in nature. The raw materials are referred to by their chemical name. Unless otherwise mentioned, the amounts indicated are percentages by weight.

### EXAMPLES

The gemini surfactant used in the examples hereinafter is a mixture of behenyl alcohol, glyceryl stearate, glyceryl stearate citrate and sodium dicocoylethylenediamine PEG-15 sulfate sold by the company Sasol under the name Ceralution® H.

### Example 1

### Anti-Aging Cream

| Phase | INCI name | % |
|---|---|---|
| A | Gemini surfactant | 3.00 |
| | Hexyldecanol/hexadecyl laurate | 5.6 |
| | Isohexadecane | 11.6 |
| | Carnauba wax | 0.75 |
| | Polymethylene wax (Cirebelle 303 sold | 8.00 |
| | by the company Cirebelle) | |
| | Beeswax | 3.00 |
| A1 | Mixture of polyglycerolated (3 mol) | 5.00 |
| | vegetable (mimosa/jojoba/sunflower) | |
| | vegetable waxes sold under the name | |
| | Hydracire S by the company Gattefosse | |
| B | Glycerol | 7.00 |
| | Preservatives | 0.6 |
| | Water | q.s. |
| | | for 100 |
| | Silicone wax sold under the name Dow | 5.00 |
| | Corning 2501 Cosmetic Wax by the | |
| | company Dow Corning | |
| C | Polyacrylamidomethylpropanesulfonic | 1.00 |
| | acid partially neutralized with aqueous | |
| | ammonia and highly crosslinked (Hostacerin | |
| | AMPS sold by the company Clariant) | |
| | Xanthan gum | 0.25 |
| | Mixture of α,ω-dihydroxylated | 1.00 |
| | polydimethylsiloxane/polydimethylsiloxane | |
| | 5 cSt (Dow Corning 1503 Fluid sold by the | |
| | company Dow Corning) | |
| | Polydimethylsiloxane | 2.50 |
| D | Modified maize starch sold under the name | 3.00 |
| | Dry Flo PC by the company National Starch | |

Manufacturing Process:

Slowly pour the hot oily phase A+A1 (80° C.) into the aqueous phase B at 80° C. in a mixer. Mix. Then disperse phase C, still in a mixer. Place the emulsion in a Rayneri mixer (deflocculator) and introduce phase D. Leave to cool with stirring.

Sensory evaluation: The texture is very thick and rich, but it applies easily while melting away on the skin and providing nutrition and immediate moisturization. It leaves the skin matt and soft.

### Example 2

### Mature-Skin Nutritive Butter Comprising the Combination of a Polar Wax (Carnauba) with One or More Apolar Oils (Isohexadecane)

| Phase | INCI name | % |
|---|---|---|
| A | Glycerol | 7.00 |
| | Disodium salt of ethylenediaminetetraacetic | 0.10 |
| | acid | |
| | Oxyethylenated stearyl alcohol (20 EO) | 1.00 |
| | Water | 22.10 |
| B | Gemini surfactant | 3.00 |
| | Carnauba wax | 2.00 |
| | Cocoa butter | 5.00 |
| | Cetyl alcohol | 4.00 |
| | Isononyl isononanoate | 5.00 |
| | Silica (Dow Corning VM-2270 Aerogel | 1.00 |
| | Fine Particles sold by the company | |
| | Dow Corning) | |
| | Polymethylene wax (Cirebelle 303 sold | 6.00 |
| | by the company Cirebelle) | |
| | Hydrogenated isoparaffin (Parleam sold | 4.00 |
| | by the company Nof Corporation) | |
| | Mixture of cetostearyl 2-ethylhexanoate/ | 4.00 |
| | isopropyl myristate (Dub Liquide IP sold | |
| | by the company Stearinerie Dubois) | |
| | Pentaerythrityl tetraisostearate | 2.00 |
| | Polydimethylsiloxane | 2.50 |
| | Caprylyl methicone | 1.50 |
| | Caprylyl glycol | 0.10 |
| | Phenoxyethanol | 0.20 |
| C | Polyacrylamidomethylpropanesulfonic acid | 0.50 |
| | partially neutralized with aqueous ammonia | |
| | and highly crosslinked (Hostacerin AMPS | |
| | sold by the company Clariant) | |
| | Xanthan gum | 0.25 |
| | Mixture of α,ω-dihydroxylated | 2.00 |
| | polydimethylsiloxane/polydimethylsiloxane | |
| | 5 cSt (Dow Corning 1503 Fluid sold by the | |
| | company Dow Corning) | |
| D | Silica | 3.00 |
| E | Water | 23.75 |

Manufacturing Process:

Slowly pour the hot oily phase B (80° C.) into the aqueous phase A at 80° C. in a mixer. Mix. Then disperse phase C, still in a mixer. Place the emulsion in a Rayneri mixer (deflocculator) and introduce phase D and then phase E. Leave to cool with stirring.

Sensory evaluation: The butter texture obtained applies easily, melts away, glides on and moisturises, and provides a matt and clean-skin effect on the skin.

### Example 3

### (Comparative) of a Composition Outside the Invention Which Contains Only Oils and Polar Waxes

| Phase | INCI name | % |
|---|---|---|
| A | Glycerol | 7.00 |
| | Disodium salt of ethylenediaminetetraacetic | 0.10 |
| | acid | |
| | Oxyethylenated stearyl alcohol (20 EO) | 1.00 |
| | Water | 22.10 |
| B | Gemini surfactant | 3.00 |
| | Carnauba wax | 2.00 |
| | Cocoa butter | 5.00 |
| | Cetyl alcohol | 4.00 |
| | Isononyl isononanoate | 7.00 |
| | Silica (Dow Corning VM-2270 Aerogel | 1.00 |
| | Fine Particles sold by the company | |
| | Dow Corning) | |
| | Candelilla wax | 6.00 |
| | Shea butter (lipex 202 sold by the | 4.00 |
| | company AarhusKarshamn) | |
| | Mixture of cetostearyl 2-ethylhexanoate/ | 4.00 |
| | isopropyl myristate | |
| | Polydimethylsiloxane 5 cSt | 2.50 |
| | Caprylyl methicone | 1.50 |
| | Caprylyl glycol | 0.10 |
| | Phenoxyethanol | 0.20 |
| C | Polyacrylamidomethylpropanesulfonic acid | 0.50 |
| | partially neutralized with aqueous ammonia | |
| | and highly crosslinked (Hostacerin AMPS | |
| | sold by the company Clariant) | |
| | Xanthan gum | 0.25 |
| | Mixture of α,ω-dihydroxylated | 2.00 |
| | polydimethylsiloxane/polydimethylsiloxane | |
| | 5 cSt (Dow Corning 1503 Fluid sold by the | |
| | company Dow Corning) | |
| D | Silica microspheres (SB 700 sold by the | 3.00 |
| | company Miyoshi Kasei) | |
| E | Water | 23.75 |

Manufacturing Process:

Slowly pour the hot oily phase (80° C.) into the aqueous phase at 80° C. in a mixer. Mix. Then disperse the ammonium polyacryloyldimethyltaurate and the xanthan gum, still in a mixer, until it feels that the product is swelling and becoming smooth. Place the emulsion in a Rayneri mixer (deflocculator) and introduce the silicones and then the fillers. Leave to cool with stirring.

Sensory evaluation: the texture is softer compared with examples 1 and 2 according to the invention; the emulsion is not structured, it has a soft consistency, it runs on application, it does not glide well and does not melt away. After application, the formula leaves a greasy and shiny film on the skin.

### Examples 4 and 5

| | | Compo- | Compo- |
|---|---|---|---|
| | | sition 4 | sition 5 * |
| Phase | INCI name | % | % |
| A | Glycerol | 7.00 | 7.00 |
| | Disodium salt of ethylenediamine- | 0.10 | 0.10 |
| | tetraacetic acid | | |
| | Water | 22.8 | 22.80 |
| B | Gemini surfactant | 5.00 | 5.00 |
| | Polymethylene wax (Cirebelle 303 | 4.00 | 14.00 |
| | sold by the company Cirebelle) | | |
| | Poly(stearyl acrylate) | 2.00 | 2.00 |
| | (Intelimer IPA 13-1 sold by the | | |
| | company Landec) | | |
| | Hydrogenated isoparaffin | 10.00 | 10.00 |
| | (Parleam sold by the company | | |
| | Nof Corporation) | | |
| | Pentaerythrityl tetraoctanoate | 10.00 | 10.00 |
| | Methyl p-hydroxybenzoate | 0.4 | 0.4 |
| | Phenoxyethanol | 0.50 | 0.50 |
| C | Polyacrylamidomethylpropane- | 0.50 | 0.50 |
| | sulfonic acid partially neutral- | | |
| | ized with aqueous ammonia and | | |
| | highly crosslinked (Hostacerin | | |
| | AMPS sold by the company | | |
| | Clariant) | | |
| | Mixture of α,ω-dihydroxylated | 7.50 | 7.50 |
| | polydimethylsiloxane/polydimethyl- | | |
| | siloxane 5 cSt (Dow Corning 1503 | | |
| | Fluid sold by the company Dow | | |
| | Corning) | | |
| D | Silica microspheres (SB 700 sold by | 3.00 | 3.00 |
| | the company Miyoshi Kasei) | | |
| E | Water | 27.1 | 17.10 |

* Composition according to the invention

Manufacturing Process:

Slowly pour the hot oily phase (80° C.) into the aqueous phase at 80° C. in a mixer. Mix. Then disperse the ammonium polyacryloyldimethyltaurate, still in a mixer, until it feels that the product is swelling and becoming smooth. Place the emulsion in a Rayneri mixer (deflocculator) and introduce the silicones and then the fillers. Leave to cool with stirring.

Sensory evaluation: the texture of the composition according to the invention is a butter which melts away, which applies very easily, it glides on, it is nourishing and moisturising and it provides a matt and clean-skin effect on the skin, unlike the comparative composition which is a non-structured creamy emulsion which leaves a greasy film on the skin.

1. Cosmetic composition of oil-in-water emulsion type comprising:
(1) at least one gemini surfactant of formula (I): 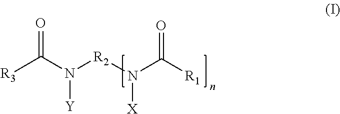
in which:
R₁ and R₃ denote, independently of one another, an alkyl radical having from 1 to 25 carbon atoms;
R₂ denotes a spacing group consisting of a linear or branched alkylene chain having from 1 to 12 carbon atoms;
X and Y denote, independently of one another, a —(C₂H₄O)ₐ—(C₃H₆O)_{b}Z group, where:
Z denotes a hydrogen atom or a —CH₂—COOM, —SO₃M, —P(O)(OM)₂, —C₂H₄—SO₃M, —C₃H₆—SO₃M or —CH₂(CHOH)₄CH₂OH radical, where M represent H or an alkali metal or alkaline earth metal or ammonium or alkanolammonium ion,
a ranges from 0 to 15,
b ranges from 0 to 10, and
the sum of a+b ranges from 1 to 25; and
n ranges from 1 to 10; and
(2) at least 20% by weight of the total weight of the composition of a fatty phase comprising at least one oil and at least one fatty substance chosen from solid fatty substances and pasty fatty substances;
the level of fatty substances chosen from solid fatty substances and pasty fatty substances representing between ⅓ and ⅔ by weight of the fatty phase.

2. Composition according to claim 1, wherein each of the R₁—CO— and R₃—CO— groups comprises from 8 to 20 carbon atoms.

3. Composition according to claim 1, wherein, for the gemini surfactant of formula (I), for each of the X and Y radicals, the sum of a and b has an average value ranging from 10 to 20.

4. Composition according to claim 1, wherein, for the gemini surfactant of formula (I), Z is the —SO₃M group, where M is an alkali metal ion.

5. Composition according to claim 1, wherein, for the gemini surfactant of formula (I), n is equal to 1.

6. Composition according to claim 1, wherein the surfactant of formula (I) has the following structure:

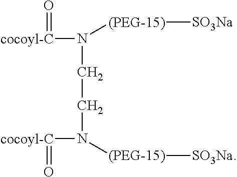

7. Composition according to claim 1, wherein the solid fatty substance(s) is (are) chosen from waxes.

8. Composition according to claim 7, wherein the wax(es) is (are) chosen from waxes, which are solid at ambient temperature, of animal, vegetable, mineral or synthetic origin, and mixtures thereof.

9. Composition according to claim 7, wherein the wax(es) is (are) chosen from candelilla wax, rice bran wax, sunflower seed wax and mixtures thereof.

10. Composition according to claim 1, wherein the pasty fatty substance(s) is (are) chosen from a mixture of hydrogenated soybean, coconut, palm and rapeseed vegetable oils, shea butter, cocoa butter, shorea butter, and mixtures thereof.

11. Composition according to claim 1, wherein the oil(s) is (are) chosen from volatile or non-volatile oils.

12. Composition according to claim 1, wherein the fatty phase comprises at least one wax, at least one pasty fatty substance, and at least one oil.

13. Composition according to claim 1, wherein the fatty phase comprises the combination either of at least one polar wax, with at least one apolar oil, or of at least one apolar wax, with a polar oil.

14. Composition according to claim 1, wherein the level of fatty substances chosen from the solid fatty substances and the pasty fatty substances is at least 10% by weight relative to the total weight of the composition.

15. Method for the cosmetic treatment of a keratin material, in which a composition as defined in claim 1 is applied to the keratin material.

16. Composition according to claim 1, wherein each of the R₁—CO— and R₃—CO— groups denotes a coconut fatty acid residue.

17. Composition according to claim 2, wherein, for the gemini surfactant of formula (I), for each of the X and Y radicals, the sum of a and b has an average value ranging from 10 to 20.

18. Composition according to claim 2, wherein, for the gemini surfactant of formula (I), Z is the —SO₃M group, where M is an alkali metal ion.

19. Composition according to claim 3, wherein, for the gemini surfactant of formula (I), Z is the —SO₃M group, where M is an alkali metal ion.

20. Composition according to claim 2, wherein, for the gemini surfactant of formula (I), n is equal to 1.
